(19) United States
(12) Patent Application Publication
(10) Pub. No.: US 2019/0119746 A1
(43) Pub. Date: Apr. 25, 2019

(54) OLIGONUCLEOTIDES FOR SELECTIVE AMPLIFICATION OF NUCLEIC ACIDS

(71) Applicant: NuGEN Technologies, Inc., San Carlos, CA (US)

(72) Inventors: Doug Amorese, Los Altos, CA (US); Benjamin G. Schroeder, San Mateo, CA (US); Nurith Kurn, Palo Alto, CA (US); Ashesh Saraiya, San Carlos, CA (US)

(21) Appl. No.: 16/164,164

(22) Filed: Oct. 18, 2018

Related U.S. Application Data
(60) Provisional application No. 62/575,051, filed on Oct. 20, 2017.

(51) Int. Cl. C12Q 1/6874; C12N 15/10

(52) U.S. Cl. CPC C12Q 1/6874; C12Q 2600/178; C12N 15/1093

(57) ABSTRACT

Provided herein are methods and compositions for selective amplification of nucleic acids. The compositions include oligonucleotides with sequence features that allow simultaneous, parallel amplification of multiple targets from a mixture of nucleic acids in a single reaction. Methods of using such oligonucleotides to identify individual targets and create libraries of targets from mixtures of nucleic acids are also provided.

### CROSS-REFERENCE TO RELATED APPLICATION

This application claims the benefit of U.S. provisional application Ser. No. 62/575,051, filed Oct. 20, 2017, incorporated by reference.

### TECHNICAL FIELD

The invention relates generally to methods and compositions for selective amplification and counting of nucleic acids.

### BACKGROUND

There is a genetic basis for many of the most-common diseases. Early detection is a critical factor in the success of treatment. Genetic biomarkers facilitate early detection. Advances in technology and clinical studies have made various types of RNA molecules, including mRNAs and miRNAs, increasingly attractive as biomarkers for disease. Moreover, compared to protein biomarkers, RNA biomarkers may provide greater sensitivity and specificity and are relatively inexpensive to analyze.

Existing methods for analyzing RNA have several shortcomings. For example, methods for sequencing miRNAs involve ligation of known sequences to the ends of RNA molecules using RNA ligase, but the efficiency of the ligation reaction is generally poor and varies depending on the sequence at end of a given miRNA. mRNAs can be sequenced by whole transcriptome shotgun sequencing, but that method results in sequence information from all transcripts—not just the diagnostically-relevant sequences. Because the target biomarkers for a given disease typically represent a small subset of the mRNA population, a disproportionate amount of the analysis time is spent discarding irrelevant mRNAs. mRNA populations can also be analyzed by hybridization to microarrays, but that approach has limited sensitivity and throughput capacity. In addition, microarrays are labor-intensive to create and cannot be readily adapted once made, so they are not well-suited for diagnosis of multiple diseases that have distinct sets of biomarker mRNAs. On the other hand, quantitative PCR (qPCR), which is more sensitive and relatively inexpensive, has a limited ability to analyze multiple mRNA biomarkers simultaneously. Consequently, analysis of even a small panel of mRNA biomarkers requires performing multiple qPCR reactions in parallel, making it impractical for most diagnoses. Due to the technical, logistical, and financial barriers of existing methods of RNA analysis, genetic diseases that could be detected at early stages go undiagnosed, and conditions like heart disease, cancer, and respiratory disease continue to kill and incapacitate millions of people each year.

### SUMMARY

The invention provides compositions and methods for amplifying from a mixture of nucleic acid molecules, such as miRNAs or mRNAs, only those molecules that contain sequences of interest. Methods of the invention selectively protect target diagnostic sequences, while allowing non-target sequences to be degraded. In one aspect, methods of the invention rely, in part, on conversion of dC into dU and subsequent degradation of dU-containing sequence and/or the inability of a dU-containing sequence to act as a template for DNA polymerase. For example, a sample containing RNA of interest is exposed to a construct comprising a sequence complementary to the target RNA and a portion that is used as a priming site for template-dependent base extension. This hybridization mixture is then treated with bisulfite, which converts unpaired cytosine bases to uracil. Next, uracil-free oligonucleotides are amplified by, for example, PCR using generic primers to create a pool of DNA molecules from the paired oligonucleotides. Thus, the pool of amplified DNA molecules includes only DNA molecules containing sequences that are represented in the collection of oligonucleotides and present in the starting population of RNA molecules. By counting species in the pool of amplified DNA molecules, the number and relative abundance of different RNA species of interest in the starting population can be determined. The invention also provides oligonucleotides for performing such methods.

In another example, a template is used that has a central region that hybridizes to a target RNA sequence. The central region is bordered by 3′ and 5′ regions on either end. The 3′ region can be any appropriate length, but preferably comprises at least about 10 bases. The 3′ region can contain a mixture of all four Watson-Crick bases or may lack Cytosine. The 5′ region generally is longer than the 3′ region but should be at least about 10 bases (so can be the same length as the 3′ region). In a preferred embodiment in which a plurality of templates is used, all 3′ regions share a common sequence. The 5′ region can also be made up of all four Watson-Crick bases but must contain Cytosine. A target RNA anneals to the central region of the template, DNA polymerase is used to conduct template-dependent base extension using the 5′ region as the template. Following extension, the sample is treated with sodium bisulfite, or an equivalent treatment such as cytosine deaminase, to convert dC residues to dU. Exposed dC residues are converted to dU but those that are double-stranded (i.e., due to base extension) are protected. RNA that has not annealed and been extended is enzymatically degraded or are unable to be amplified in subsequent PCR. Only the protected templates will remain and then are analyzed.

As an alternative, the invention contemplates the use of stem-loop structures that have a target-specific loop region and universal (i.e., common) stem sequences that contain universal priming sites for PCR and a restriction site in the complementary (double-stranded) portion of the stem. The stem-loop constructs are annealed to the target and exposed to a restriction enzyme that attacks the common restriction site in the complementary portions of the stem. The restriction enzyme removes the priming sites from any unpaired stem-loop structures. In the presence of target, the stem-loop probe is protected from the restriction enzyme and thus can be amplified. Thus, targets of interest are protected and are selectively amplified and pulled out of the sample.

Because compositions and methods of the invention allow selective amplification of nucleic acids of interest, they are useful for diagnosing diseases associated with genetic alterations, such as heart disease, cancer, and respiratory disease. Claimed methods expedite diagnostic screening by eliminating the need to sift through irrelevant information. Another advantage of methods of the invention is that the use of generic PCR primers to amplify different RNA species permits a large number of RNA species to be amplified in the same reaction. Consequently, an entire set of biomarkers for a given disease can be analyzed in a single assay. In addition, different RNA species in the starting population are represented in the amplified pool of DNA molecules in an unbiased, sequence-independent manner, which allows the methods to detect small differences in number and abundance of different RNA molecules within a set of biomarkers.

In an aspect, the invention provides methods of identifying a target in a mixture of nucleic acid molecules, the methods including the following steps: providing a mixture of nucleic acid molecules; adding to the mixture a non-naturally-occurring oligonucleotide that includes a 5′ region containing at least one cytosine, a central region complementary to a portion of the target, and a 3′ region, such that the oligonucleotide anneals to the target; converting unpaired cytosines in the mixture to uracil; and detecting the oligonucleotide, thereby identifying the target in the mixture. Methods may include extending the 3′ end of the annealed target using the 5′ region of the oligonucleotide as a template.

The nucleic acids in the mixture may be single-stranded or double-stranded. If the nucleic acids of the mixture are provided as double-stranded, methods of the invention may include a denaturation step.

Constructs for use in the invention may be DNA, RNA, or a mixed nucleic acid containing both ribonucleotides and deoxyribonucleotides. For DNA oligonucleotides, the 3′ region may contain the four bases that occur naturally in DNA, i.e., adenine, cytosine, guanine, and thymine, or the 3′ region may be free of cytosine and/or may only contain adenine, guanine, and thymine.

Target nucleic acid molecules may be DNA, RNA, or a mixed nucleic acid containing both ribonucleotides and deoxyribonucleotides. The RNA molecules may be mRNA, miRNA, piRNA, siRNA, shRNA, tRNA, rRNA, snRNA, or snoRNA.

Detection includes amplifying nucleic acid molecules, e.g., DNA or RNA, that do not contain uracil. Nucleic acid molecules may be amplified by PCR. PCR primers may contain sequences that are identical to or complementary to sequences in the 5′ region and 3′ region of the oligonucleotide. Detection may include degrading nucleic acid molecules, e.g., DNA or RNA, that contain uracil. Degradation may include treatment of the mixture with a uracil-DNA glycosylase, DNA exonuclease, DNA AP lyase, heat, or alkaline conditions.

In another aspect, the invention provides methods of identifying a target in a mixture of nucleic acid molecules, including the following steps: providing a mixture of nucleic acid molecules; adding to the mixture a non-naturally-occurring oligonucleotide that includes a 5′ region free of cytosines, a central region that contains one or more cytosines and is complementary to a portion of the target, and a 3′ region free of cytosines, such that the oligonucleotide anneals to the target, thereby forming one or more base pairs between one or more cytosines in the central region of the oligonucleotide and one or more guanines in the target; converting unpaired cytosines in the mixture to uracil; and detecting the oligonucleotide, thereby identifying the target in the mixture.

In another aspect, the invention provides non-naturally-occurring oligonucleotides for identifying a target from a mixture of nucleic acid molecules, the oligonucleotides including a 5′ region that contains at least one cytosine, a central region complementary to a portion of the target nucleic acid molecule, and a 3′ region.

In another aspect, the invention provides non-naturally-occurring oligonucleotides for identifying a target from a mixture of nucleic acid molecules, the oligonucleotide including a 5′ region free of cytosines, a central region that contains at least one cytosine and is complementary to a portion of the target nucleic acid molecule, and a 3′ region free of cytosines.

In another aspect, the invention provides methods of making a library of targets from a mixture of RNA molecules, the methods including the following steps: providing a mixture of RNA molecules; adding to the mixture multiple non-naturally-occurring oligonucleotides, each oligonucleotide containing a common 5′ region that contains one or more cytosines, a central region complementary to a portion of a target, and a common 3′ region, such that one or more of the oligonucleotides anneal with targets; converting unpaired cytosines in the mixture to uracil; and selecting oligonucleotides that do not contain uracil for making the nucleic acid library.

In another aspect, the invention provides methods of making a library of targets from a mixture of RNA molecules, the methods including the following steps: providing a mixture of RNA molecules; adding to the mixture multiple non-naturally-occurring oligonucleotides, each oligonucleotide containing a common 5′ region free of cytosines, a central region that contains one or more cytosines and is complementary to a portion of a target, and a common 3′ region free of cytosines, such that one or more oligonucleotides anneal with targets, thereby forming base pairs between one or more cytosines in the central region of the oligonucleotide and one or more guanines in the target; converting unpaired cytosines in the mixture to uracil; and selecting oligonucleotides that do not contain uracil for making the nucleic acid library. Preferably, unpaired cytosines are converted to uracil by adding bisulfite ions to the mixture.

In another aspect, the invention provides collections of non-naturally-occurring oligonucleotides for making a library of targets from a mixture of RNA molecules, each oligonucleotide including a common 5′ region containing at least one cytosine, a central region complementary to a portion a target; and a common 3′ region.

In another aspect, the invention provides collections of non-naturally-occurring oligonucleotides for making a library of targets from a mixture of RNA molecules, each oligonucleotide including a common 5′ region free of cytosines, a central region that contains at least one cytosine and is complementary to a portion of a target, and a common 3′ region free of cytosines.

Other aspects and advantages of the invention are provided below in the detailed description thereof.

### BRIEF DESCRIPTION OF THE DRAWINGS

FIG. 1 is a schematic of a non-naturally oligonucleotide according to an embodiment of the invention.

FIG. 2 is a schematic of a non-naturally oligonucleotide according to an embodiment of the invention.

FIG. 3 illustrates a method of using an oligonucleotide to identify a target in a mixture of nucleic acids according to an embodiment of the invention.

FIG. 4 illustrates a method of using an oligonucleotide to identify a target in a mixture of nucleic acids according to an embodiment of the invention.

FIG. 5 illustrates and embodiment of the invention in which stem-loop structures are used in conjunction with restriction enzymes to selectively protect target sequence.

### DETAILED DESCRIPTION

The invention provides methods and compositions for selective amplification of one or more targets from a mixture of nucleic molecules. For example, RNA may be selectively amplified from a biological sample. Methods and compositions provided herein allow multiple species to be amplified simultaneously from a nucleic acid mixture, obviating the need to perform multiple parallel amplification steps to analyze a group of targets. In addition, the amplified material includes only molecules of interest, which typically comprise a small subset of molecules present in the starting material. Consequently, the methods and compositions streamline downstream analysis by eliminating the need to examine a vast number of uninformative species.

Compositions of the invention include oligonucleotides that allow selective amplification of targets from a mixture of single-stranded nucleic molecules. The oligonucleotides contain strategically-positioned cytosine bases that become paired with complementary guanine bases when the oligonucleotides anneal to their targets. One or more of such cytosines, however, remain unpaired when the oligonucleotides are imperfectly matched with an off-target species or do not hybridize to a nucleic acid at all. After oligonucleotides are allowed to hybridize with nucleic acids in the mixture, the mixture is treated to convert unpaired cytosines to uracil but leave paired cytosines intact. This may be achieve with bisulfite ions or with cytosine deaminase. Uracil-free oligonucleotides are then amplified by the polymerase chain reaction (PCR) to obtain a collection of targets that is free from irrelevant species.

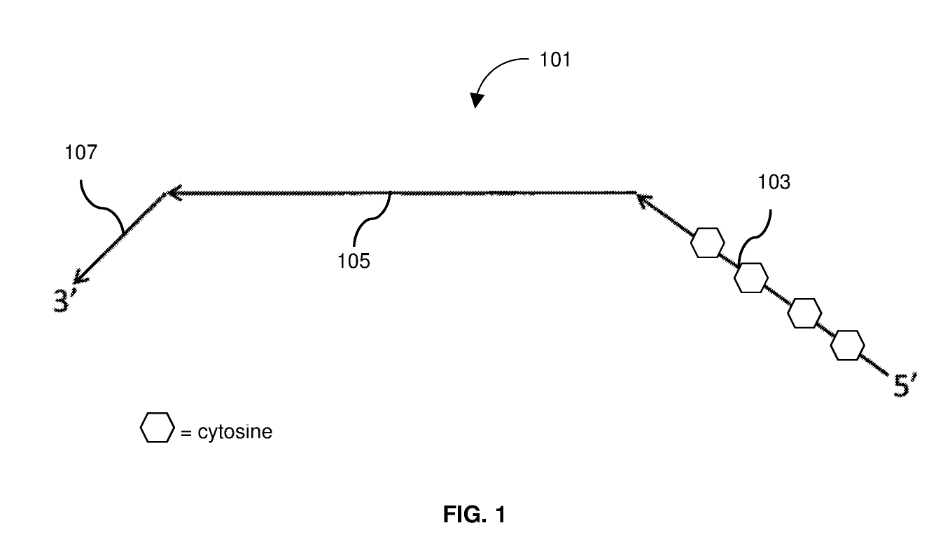

FIG. 1 is a schematic of a non-naturally oligonucleotide 101 according to an embodiment of the invention. The oligonucleotide 101 includes a 5′ region 103 that contains at least one cytosine, a central region 105 complementary to a portion of the target nucleic acid molecule, and a 3′ region 107. The oligonucleotide 101 may be DNA, RNA, or a mixed nucleic acid containing both ribonucleotides and deoxyribonucleotides. Preferably, the oligonucleotide 101 is DNA.

The 5′ region 103 of the oligonucleotide 101 may be 10-20 nucleotides in length or longer. For example and without limitation, the 5′ region 103 may contain at least 8 nucleotides, at least 10 nucleotides, at least 12 nucleotides, at least 15 nucleotides, at least 25 nucleotides, at least 30 nucleotides, or at least 40 nucleotides. The 5′ region 105 contains at least one cytosine, and it may contain at least 2 cytosines, at least 3 cytosines, at least 4 cytosines, at least 5 cytosines, or at least 6 cytosines.

The 3′ region 107 of the oligonucleotide 101 may be as short as 10-15 nucleotides in length. For example and without limitation, the 3′ region 107 may contain at least 8 nucleotides, at least 10 nucleotides, at least 12 nucleotides, at least 15 nucleotides, at least 25 nucleotides, at least 30 nucleotides, or at least 40 nucleotides. For DNA oligonucleotides, the 3′ region 107 may contain the four bases that occur naturally in DNA, i.e., adenine, cytosine, guanine, and thymine, or it may contain only adenine, guanine, and thymine.

The central region 105 of the oligonucleotide 101 is complementary to a portion of the target nucleic acid molecule. Complementarity refers to the ability of a single strand of a nucleic acid to form base pairs with another strand of a nucleic acid in an antiparallel manner. Generally, base pairs are formed between adenine and thymine, between adenine and uracil, and between cytosine and guanine. However, adenine can form base pairs with guanine, and the central region may contain one or more adenines that form base pairs with guanines in the complementary region of the target.

The central region 105 may contain one or more portions that are free of cytosines. The cytosine-free portion may be at the 5′ portion of the central region 105 or the 3′ portion of the central region 105. The cytosine-free portion may contain 10 or fewer nucleotides, 9 or fewer nucleotides, 8 or fewer nucleotides, 7 or fewer nucleotides, 6 or fewer nucleotides, 5 or fewer nucleotides, 4 or fewer nucleotides, 3 or fewer nucleotides, or 2 or fewer nucleotides. The cytosine-free portion of the central region 105 may contain one or more adenines that form base pairs with guanines in the complementary region of the target.

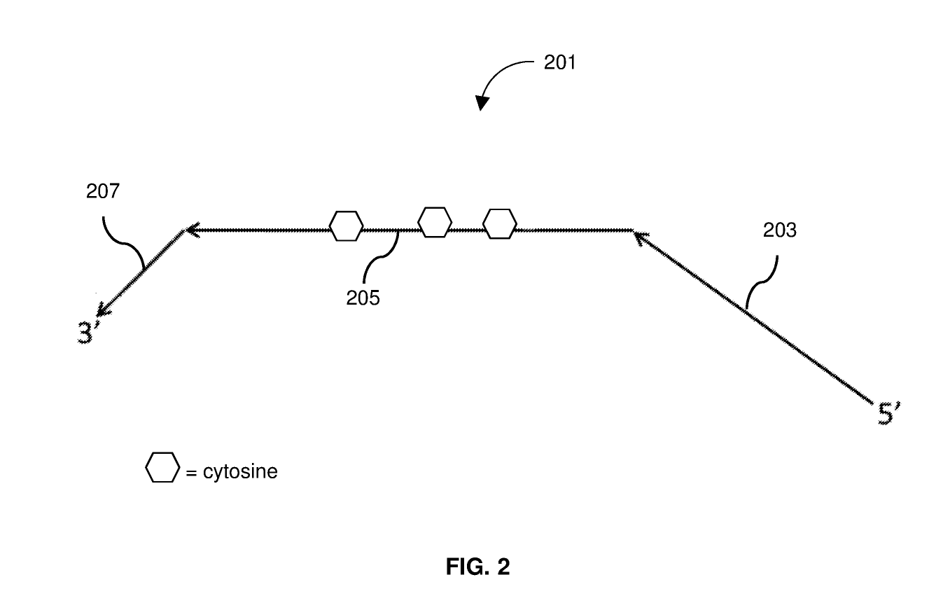

FIG. 2 is a schematic of a non-naturally oligonucleotide 201 according to an embodiment of the invention. The oligonucleotide 201 includes a 5′ region 203 free of cytosines, a central region 205 that contains at least one cytosine and is complementary to a portion of the target nucleic acid molecule, and a 3′ region 207 free of cytosines. The oligonucleotide 201 may be DNA, RNA, or a mixed nucleic acid containing both ribonucleotides and deoxyribonucleotides. Preferably, the oligonucleotide 201 is DNA.

The 5′ region 203 of the oligonucleotide 201 may be as short as 10-15 nucleotides in length. For example and without limitation, the 5′ region 203 may contain at least 8 nucleotides, at least 10 nucleotides, at least 12 nucleotides, at least 15 nucleotides, at least 25 nucleotides, at least 30 nucleotides, or at least 40 nucleotides. For DNA oligonucleotides, the 5′ region 203 may contain any sequence or combination of adenine, guanine, and thymine.

The 3′ region 207 of the oligonucleotide 201 may be as short as 10-15 nucleotides in length. For example and without limitation, the 3′ region 207 may contain at least 8 nucleotides, at least 10 nucleotides, at least 12 nucleotides, at least 15 nucleotides, at least 25 nucleotides, at least 30 nucleotides, or at least 40 nucleotides.

The central region 205 of the oligonucleotide 201 contains at least one cytosine, and it may contain at least 2 cytosines, at least 3 cytosines, at least 4 cytosines, at least 5 cytosines, or at least 6 cytosines. However, the central region 205 may contain one or more portions that are free of cytosines. The cytosine-free portion may be at the 5′ portion of the central region 205 or the 3′ portion of the central region 205. The cytosine-free portion may contain 10 or fewer nucleotides, 9 or fewer nucleotides, 8 or fewer nucleotides, 7 or fewer nucleotides, 6 or fewer nucleotides, 5 or fewer nucleotides, 4 or fewer nucleotides, 3 or fewer nucleotides, or 2 or fewer nucleotides. The cytosine-free portion of the central region 205 may contain one or more adenines that form base pairs with guanines in the complementary region of the target.

In another aspect, the invention provides methods of using the oligonucleotides described above to identify a target in a mixture of nucleic acid molecules.

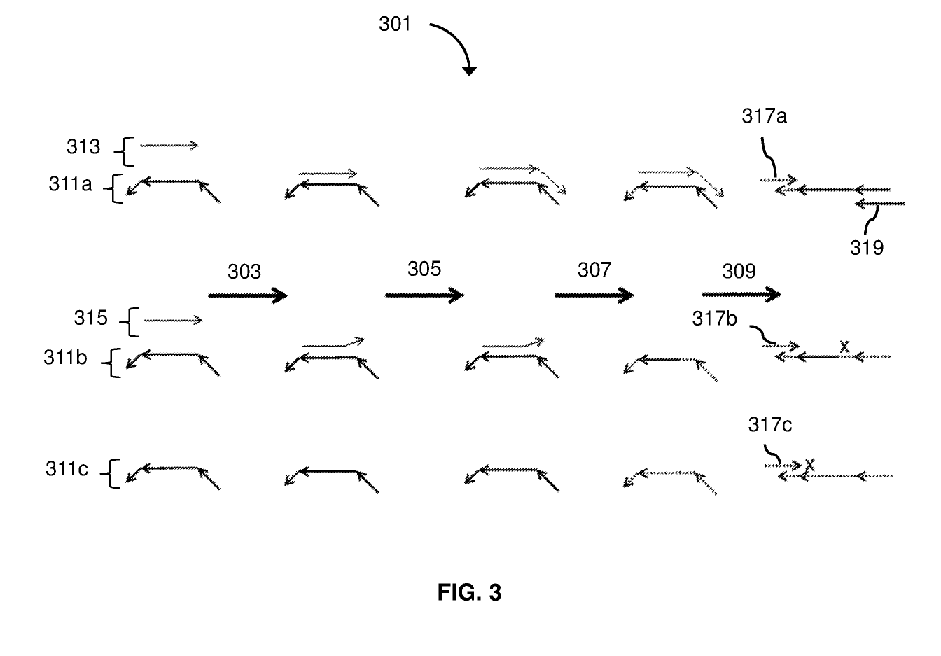

FIG. 3 illustrates a method 301 of using an oligonucleotide to identify a target in a mixture of nucleic acids according to an embodiment of the invention. In this embodiment, the oligonucleotide includes the features described above for the oligonucleotide 101. The method 301 is particularly useful for identification of a target in a mixture of miRNA.

In a first step 303, the oligonucleotide is added to a mixture of single-stranded nucleic acids and allowed to anneal to nucleic acids in the mixture. A copy 311a of the oligonucleotide hybridizes with the target 313 nucleic acid based on the complementarity between the central region of the oligonucleotide and a portion of the target 313. A copy 311b of the oligonucleotide partially hybridizes with an off-target 315 nucleic acid due to partial complementarity between the oligonucleotide and the off-target 315 nucleic acid. A copy 311c of the oligonucleotide fails to hybridize to a nucleic acid in the mixture.

The single-stranded nucleic acids may be DNA, RNA, or mixed nucleic acid containing both ribonucleotides and deoxyribonucleotides. The nucleic acids may be provided as double-stranded molecules and then denatured. Thus, in some embodiments, the methods include a denaturation step. Denaturation may be achieved by heating, changing the pH, or any other means known in the art. In embodiments in which the nucleic acid is RNA, the RNA may be a type or sub-class of RNA. For example and without limitation, the RNA may be mRNA, miRNA, piRNA, siRNA, shRNA, tRNA, rRNA, snRNA, or snoRNA.

The methods may include an extension step 305 in which the 3′ end of the target 313 nucleic acid is extended using the 5′ region of the annealed copy 311a of the oligonucleotide as a template. Extension 305 may be performed using any suitable RNA polymerase or DNA polymerase. The 3′ end of the off-target nucleic acid 315 is not extended due to imperfect complementarity between the annealed copy 311b of the oligonucleotide and the off-target 315 nucleic acid.

In another step 307, unpaired cytosines in the oligonucleotide are converted to uracil. Preferably, conversion 307 is achieved by adding bisulfite ions to the mixture, which results in deamination of cytosines. Bisulfite ions may be provided as one or more salts of sodium, lithium, potassium, ammonium, tetraalkylammonium, magnesium, manganese, or calcium. The bisulfite salt may be provided as a solid, solution, gel, or any other form known in the art.

As described above in relation to the oligonucleotide 101, the 5′ region of the oligonucleotide includes one or more cytosines, and the central region of the oligonucleotide may include one or more cytosines. Cytosines in the copy 311a of the oligonucleotide annealed to the target 313 nucleic are base-paired and thus protected from bisulfite deamination. However, one or more cytosines in the 5′ region of the copy 311b of the oligonucleotide bound to the off-target nucleic acid are exposed to bisulfite and thus become deaminated; unpaired cytosines that may exist in the central region of the copy 311b of the oligonucleotide become deaminated as well. Similarly, cytosines in the copy 311c of the oligonucleotide that did not hybridize with a nucleic acid also become deaminated.

Formation of stable double-stranded duplexes facilitates protection of cytosines from oxidation. Stability of double-stranded nucleic acid duplexes, which can be inferred from the melting temperature of the duplex, depends largely on the length of the region of complementarity. For example, miRNAs typically contain about 22 nucleotides, and even an oligonucleotide having a central region that is perfectly complementary to its target has a relatively low melting temperature. However, by using an oligonucleotide with a 5′ region of 20 nucleotides, the length of double-stranded complementarity is nearly doubled after the extension step 305, which elevates the melting temperature of the duplex and provides better protection of base-paired cytosines. Consequently, for detection of small nucleic acids, such as miRNAs, it is advantageous to include an extension step 305 and to use an oligonucleotide that has a relatively long 5′ region, e.g., one that includes 20 or more nucleotides.

In another step 309, the copy 311a of the oligonucleotide that hybridized with the target 313 nucleic acid is selectively amplified by PCR. A first PCR primer 317 is complementary to a sequence in the 3′ region of the oligonucleotide, and a second PCR primer 319 is complementary to a sequence in the 5′ region of the oligonucleotide.

Selective amplification may be achieved by various methods, including combinations of methods. In some methods, a polymerase, such as a thermostable DNA polymerase, that cannot use uracil as a template is used for PCR amplification. A copy 317a of the first primer anneals to the 3′ region of the copy 311a of the oligonucleotide that was hybridized to the target 313 nucleic acid; another copy 317b of the first primer anneals to the 3′ region of the copy 311b of the oligonucleotide that was partially hybridized to the off-target 315 nucleic acid; and another copy 317c of the first primer anneals to the copy 311c of the oligonucleotide that did not hybridize to a nucleic acid. The polymerase is able to synthesize a full-length complementary strand using copy 317a as a primer and copy 311a as a template because copy 311a of the oligonucleotide contains no uracil. In contrast, the polymerase stalls during extension from copies 317b and 317c of the first primer because copies 311b and 311c of the oligonucleotide include uracil bases, and therefore the polymerase is not able to synthesize full-length complementary strands for copies 311b and 311c of the oligonucleotide.

Other methods of selective amplification include degrading uracil-containing DNA. For example, mixtures may be treated with uracil DNA glycosylase, which excises uracil from DNA strands. Mixtures may then be treated with DNA-(apurinic or apyrimidinic) lyase (AP lyase), which severs the sugar-phosphate backbone of a DNA strand that lacks a base. Additionally or alternatively, after uracil DNA glycosylase treatment, mixtures may be exposed to heat and/or alkaline conditions to cleave DNA at abasic sites.

The methods may include an RNA degradation step. The RNA degradation step may include treating the mixture with a ribonuclease (RNase). For example and without limitation, the RNase may be RNase A, RNase H, RNase III, RNase L, RNase P, RNase PhyM, RNase T1, RNase T2, RNase U2, or RNase V.

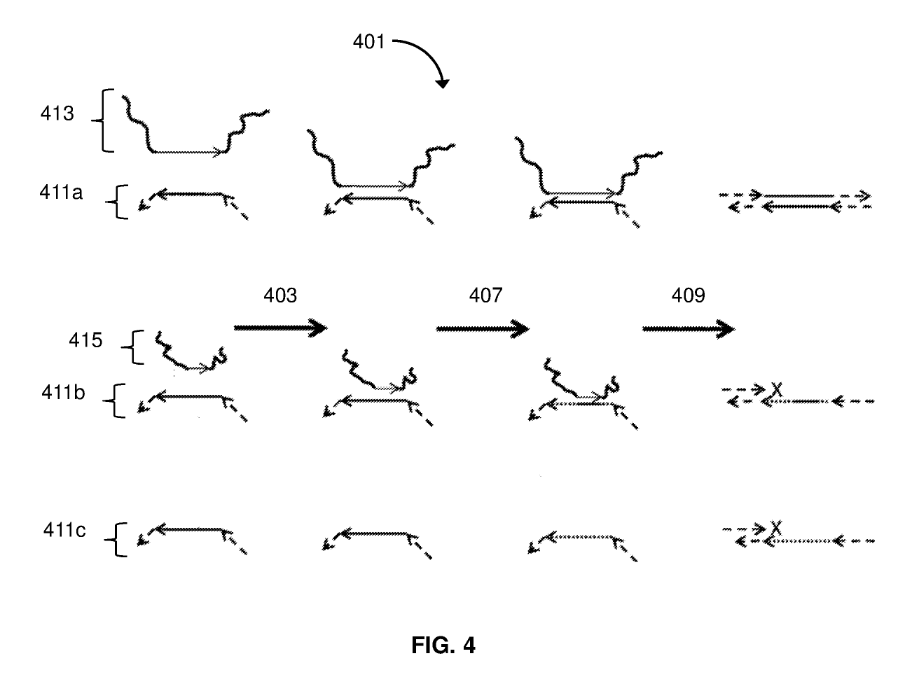

FIG. 4 illustrates a method 401 of using an oligonucleotide to identify a target in a mixture of nucleic acids according to an embodiment of the invention. In this embodiment, the oligonucleotide includes the features described above for the oligonucleotide 201. The method 401 is particularly useful for identification of a target in a mixture of mRNA.

In a first step 403, the oligonucleotide is added to a mixture of single-stranded nucleic acids and allowed to anneal to nucleic acids in the mixture. A copy 411a of the oligonucleotide hybridizes with the target 413 nucleic acid based on the complementarity between the central region of the oligonucleotide and a portion of the target 413. A copy 411b of the oligonucleotide partially hybridizes with an off-target 415 nucleic acid due to partial complementarity between the oligonucleotide and the off-target 415 nucleic acid. A copy 411c of the oligonucleotide fails to hybridize to a nucleic acid in the mixture.

The single-stranded nucleic acids may be any type of nucleic acid, as described above in relation to the method 301. The nucleic acid may be provided as double-stranded molecules and denatured, as described above in relation to the method 301.

In another step 407, unpaired cytosines in the oligonucleotide are converted to uracil. Conversion 407 may be achieved by adding bisulfite ions to the mixture, as described above in relation to the method 301.

As described above in relation to the oligonucleotide 201, the 5′ and 3′ regions of the oligonucleotide do not contain cytosines, and the central region of the oligonucleotide includes one or more cytosines. Cytosines in the copy 411a of the oligonucleotide annealed to the target 413 nucleic are base-paired and thus protected from bisulfite deamination. However, one or more cytosines in the central region of the copy 411b of the oligonucleotide bound to the off-target nucleic acid are exposed to bisulfite and thus become deaminated. Similarly, cytosines in the copy 411c of the oligonucleotide that did not hybridize with a nucleic acid also become deaminated.

In another step 409, the copy 411a of the oligonucleotide that hybridized with the target 413 nucleic acid is selectively amplified by PCR. A first PCR primer is complementary to a sequence in the 3′ region of the oligonucleotide, and a second PCR primer is complementary to a sequence in the 5′ region of the oligonucleotide. Selective amplification may be achieved by various methods, as described above in relation to the method 301.

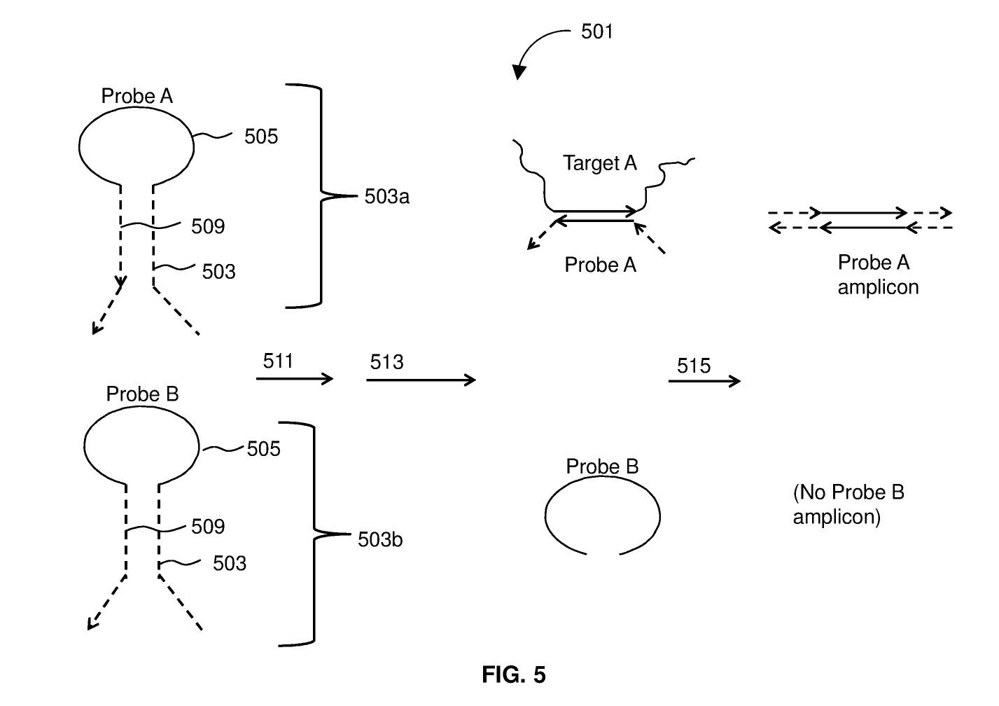

FIG. 5 shows and embodiment 501 of the invention in which a stem-loop structure is used as a probe to capture target sequence. The stem-loop structures 503a, 503b have a target-specific loop region 505 and universal (i.e., common) stem sequences 507 that contain universal priming sites for PCR and a restriction site 509 in the complementary (double-stranded) portion of the stem. The stem-loop constructs are annealed 511 to the target and exposed 513 to a restriction enzyme that attacks the common restriction site in the complementary portions of the stem. The restriction enzyme removes the priming sites from any unpaired stem-loop structures. In the presence of target, the stem-loop probe is protected from the restriction enzyme and thus can be amplified. Thus, targets of interest are protected and are selectively amplified 515 and pulled out of the sample. As shown in the Figure, Probe A hybridizes to the target so the duplex stem can't form, thus protecting the probe from the restriction enzyme and allowing it to be amplified.

In another aspect, the invention provides collections of non-naturally-occurring oligonucleotides for making a library of targets from a mixture of RNA molecules. The collections may include oligonucleotides having the same structure as the oligonucleotide 101: each oligonucleotide includes a common 5′ region containing at least one cytosine, a central region complementary to a portion a target, and a common 3′ region.

The 5′ region of the oligonucleotides of the collection may be 10-20 nucleotides in length or longer. For example and without limitation, the 5′ region may contain at least 8 nucleotides, at least 10 nucleotides, at least 12 nucleotides, at least 15 nucleotides, at least 25 nucleotides, at least 30 nucleotides, or at least 40 nucleotides. The 5′ region contains at least one cytosine, and it may contain at least 2 cytosines, at least 3 cytosines, at least 4 cytosines, at least 5 cytosines, or at least 6 cytosines.

The 3′ regions of the oligonucleotides of the collection may be as short as 10-15 nucleotides in length. For example and without limitation, the 3′ region may contain at least 8 nucleotides, at least 10 nucleotides, at least 12 nucleotides, at least 15 nucleotides, at least 25 nucleotides, at least 30 nucleotides, or at least 40 nucleotides. For DNA oligonucleotides, the 3′ region may contain the four bases that occur naturally in DNA, i.e., adenine, cytosine, guanine, and thymine, or it may contain only adenine, guanine, and thymine.

The central regions of the oligonucleotides of the collection are complementary to portions of target nucleic acid molecules. Each oligonucleotide in a collection may have a different central region. Each oligonucleotide in a collection may have a central region complementary to a different portion of a target. Each oligonucleotide in a collection may have a central region complementary to a portion of a different target.

The central regions may contain one or more portions that are free of cytosines. The cytosine-free portion may be at the 5′ portion of the central region or the 3′ portion of the central region. The cytosine-free portion may contain 10 or fewer nucleotides, 9 or fewer nucleotides, 8 or fewer nucleotides, 7 or fewer nucleotides, 6 or fewer nucleotides, 5 or fewer nucleotides, 4 or fewer nucleotides, 3 or fewer nucleotides, or 2 or fewer nucleotides. The cytosine-free portion of the central regions may contain one or more adenines that form base pairs with guanines in the complementary region of the target.

The collection of oligonucleotides may be designed to identify targets related to a disease or medical condition. For example, the collection of oligonucleotides may be used to identify biomarkers for a genetically-based disease, such as heart disease, cancer, or respiratory disease. The collection of oligonucleotides may be used to identify genetic alterations, including substitutions, insertions, deletions, truncations, single nucleotide polymorphisms, changes in copy number, changes in expression, and the like.

In another aspect, the invention provides collections of non-naturally-occurring oligonucleotides for making a library of targets from a mixture of RNA molecules. The collections may include oligonucleotides having the same structure as the oligonucleotide 201: each oligonucleotide includes common 5′ and 3′ regions that are free of cytosines and a central region that contains one or more cytosines and is complementary to a portion of a target.

The 5′ region of the oligonucleotides of the collection may be as short as 10-15 nucleotides in length. For example and without limitation, the 5′ region may contain at least 8 nucleotides, at least 10 nucleotides, at least 12 nucleotides, at least 15 nucleotides, at least 25 nucleotides, at least 30 nucleotides, or at least 40 nucleotides. For DNA oligonucleotides, the 5′ region may contain any sequence or combination of adenine, guanine, and thymine.

The 3′ region of the oligonucleotides of the collection may be as short as 10-15 nucleotides in length. For example and without limitation, the 3′ region may contain at least 8 nucleotides, at least 10 nucleotides, at least 12 nucleotides, at least 15 nucleotides, at least 25 nucleotides, at least 30 nucleotides, or at least 40 nucleotides.

The central regions of the oligonucleotides of the collection are complementary to portions of target nucleic acid molecules. Each oligonucleotide in a collection may have a different central region. Each oligonucleotide in a collection may have a central region complementary to a different portion of a target. Each oligonucleotide in a collection may have a central region complementary to a portion of a different target.

The central regions of the oligonucleotides of the collection contain at least one cytosine, and they may contain at least 2 cytosines, at least 3 cytosines, at least 4 cytosines, at least 5 cytosines, or at least 6 cytosines. However, the central regions may contain one or more portions that are free of cytosines. The cytosine-free portions may be at the 5′ portion of the central regions or the 3′ portion of the central regions. The cytosine-free portions may contain 10 or fewer nucleotides, 9 or fewer nucleotides, 8 or fewer nucleotides, 7 or fewer nucleotides, 6 or fewer nucleotides, 5 or fewer nucleotides, 4 or fewer nucleotides, 3 or fewer nucleotides, or 2 or fewer nucleotides. The cytosine-free portions of the central region may contain one or more adenines that form base pairs with guanines in the complementary regions of the targets.

The collection of oligonucleotides may be designed to identify targets related to a disease or medical condition. For example, the collection of oligonucleotides may be used to identify biomarkers for a genetically-based disease, such as heart disease, cancer, or respiratory disease. The collection of oligonucleotides may be used to identify genetic alterations, including substitutions, insertions, deletions, truncations, single nucleotide polymorphisms, changes in copy number, changes in expression, and the like.

In another aspect, the invention provides methods of making a library of targets from a mixture of RNA molecules using the collections of oligonucleotides based on the oligonucleotide 101, as described above. Such methods are based on the method 301 of identifying a target from a mixture of nucleic acids.

The methods include providing a mixture of RNA molecules. The RNA may be mRNA, miRNA, piRNA, siRNA, shRNA, tRNA, rRNA, snRNA, or snoRNA. Preferably, the RNA is miRNA.

The methods include adding to the mixture a collection of oligonucleotides in which each oligonucleotide contains a common 5′ region that contains one or more cytosines, a central region complementary to a portion of a target, and a common 3′ region, such that one or more of the oligonucleotides anneal with targets. The central regions of the oligonucleotides of the collection are complementary to portions of target nucleic acid molecules. Each oligonucleotide in a collection may have a different central region. Each oligonucleotide in a collection may have a central region complementary to a different portion of a target. Each oligonucleotide in a collection may have a central region complementary to a portion of a different target.

The methods include converting unpaired cytosines in the mixture to uracil. Conversion may be achieved by adding bisulfite ions to the mixture, as described above in relation to the method 301.

The methods include selecting oligonucleotides that do not contain uracil for making the nucleic acid library. Selection may be performed by selectively PCR amplifying oligonucleotides in the collection that have hybridized to targets, as described above in relation to the method 301.

The methods may include extending the 3′ ends of the annealed targets using the 5′ regions of the oligonucleotides as templates. Extension may be performed as described above in relation to the method 301.

The oligonucleotide may be DNA, RNA, or a mixed nucleic acid containing both ribonucleotides and deoxyribonucleotides.

The methods may include counting targets in the library. For example and without limitation, counting may include any of the following: counting the total number of amplified targets; comparing the number of targets amplified to the number of species of oligonucleotides added to the mixture; comparing the absolute or relative abundance of different amplified targets; and counting the number of target products that arose from independent amplification of the same target.

The methods may include sequencing targets in the library. The methods may include detecting changes biomarkers for a disease or medical condition. For example, the methods may include identifying genetic alterations, including substitutions, insertions, deletions, truncations, single nucleotide polymorphisms, changes in copy number, changes in expression, or the like.

The methods may include providing a diagnosis, prognosis, or course of treatment for a disease or medical condition.

In another aspect, the invention provides methods of making a library of targets from a mixture of RNA molecules using collections of oligonucleotides based on the oligonucleotide 201, as described above. Such methods are based on the method 401 of identifying a target from a mixture of nucleic acids.

The methods include providing a mixture of RNA molecules. The RNA may be mRNA, miRNA, piRNA, siRNA, shRNA, tRNA, rRNA, snRNA, or snoRNA. Preferably, the RNA is mRNA.

The methods include adding to the mixture a collection of oligonucleotides in which each oligonucleotide contains common 5′ and 3′ regions that are free of cytosines and a central region that contains one or more cytosines and is complementary to a portion of a target nucleic acid molecule, such that one or more of the oligonucleotides anneal with targets. Each oligonucleotide in a collection may have a different central region. Each oligonucleotide in a collection may have a central region complementary to a different portion of a target. Each oligonucleotide in a collection may have a central region complementary to a portion of a different target.

The methods include converting unpaired cytosines in the mixture to uracil. Conversion may be achieved by adding bisulfite ions to the mixture, as described above in relation to the method 301.

The methods include selecting oligonucleotides that do not contain uracil for making the nucleic acid library. Selection may be performed by selectively PCR amplifying oligonucleotides in the collection that have hybridized to targets, as described above in relation to the method 301.

The oligonucleotide may be DNA, RNA, or a mixed nucleic acid containing both ribonucleotides and deoxyribonucleotides.

The methods may include counting targets in the library. For example and without limitation, counting may include any of the following: counting the total number of amplified targets; comparing the number of targets amplified to the number of species of oligonucleotides added to the mixture; comparing the absolute or relative abundance of different amplified targets; and counting the number of target products that arose from independent amplification of the same target.

The methods may include sequencing targets in the library. The methods may include detecting changes biomarkers for a disease or medical condition. For example, the methods may include identifying genetic alterations, including substitutions, insertions, deletions, truncations, single nucleotide polymorphisms, changes in copy number, changes in expression, or the like.

The methods may include providing a diagnosis, prognosis, or course of treatment for a disease or medical condition.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of identifying a target in a mixture of nucleic acid molecules, the method comprising:
providing the mixture of nucleic acid molecules;
adding to the mixture a non-naturally-occurring oligonucleotide comprising:
a 5′ region comprising at least one cytosine;
a central region complementary to a portion of the target; and
a 3′ region,
such that the oligonucleotide anneals to the target;
converting unpaired cytosines in the mixture to uracil; and
detecting the oligonucleotide, thereby identifying the target in the mixture.

2. The method of claim 1, further comprising:
extending a 3′ end of the annealed target using the 5′ region of the oligonucleotide as a template.

3. The method of claim 1, wherein the converting step is selected from the group consisting of adding bisulfite ions to the mixture and exposing the mixture to cytosine deaminase.

4. The method of claim 1, wherein the oligonucleotide is DNA.

5. The method of claim 4, wherein the detecting step comprises amplifying DNA molecules that do not contain uracil.

6. The method of claim 4, wherein the detecting step comprises degrading DNA molecules that contain uracil.

7. The method of claim 1, wherein the nucleic acid molecules are miRNA.

8. A method of making a library of targets from a mixture of RNA molecules, the method comprising:
providing the mixture of RNA molecules;
adding to the mixture a plurality of non-naturally-occurring oligonucleotides, each oligonucleotide comprising:
a common 5′ region comprising at least one cytosine;
a unique central region complementary to a portion of the target; and
a common 3′ region,
such that at least one of the plurality of oligonucleotides anneals with at least one target;
converting unpaired cytosines in the mixture to uracil; and
selecting oligonucleotides that do not contain uracil for making the library.

9. The method of claim 8, further comprising:
extending a 3′ end of the annealed target using the 5′ region of the at least one of the plurality of oligonucleotides as a template.

10. The method of claim 8, wherein the converting step is selected from adding bisulfite ions to the mixture and exposing the mixture to cytosine deaminase.

11. The method of claim 8, wherein the oligonucleotides are DNA.

12. The method of claim 11, wherein the selecting comprises amplifying DNA molecules that do not contain uracil.

13. The method of claim 11, wherein the selecting step comprises degrading DNA molecules that contain uracil.

14. The method of claim 8, wherein the RNA molecules are miRNA.

15. A method of identifying a target in a mixture of nucleic acid molecules, the method comprising:
providing the mixture of nucleic acid molecules;
adding to the mixture a non-naturally-occurring oligonucleotide comprising:
a 5′ region free of cytosines;
a central region comprising at least one cytosine and complementary to a portion of the target; and
a 3′ region free of cytosines,
such that the oligonucleotide anneals to the target, thereby forming a base pair between the at least one cytosine and at least one guanine in the target;
converting unpaired cytosines in the mixture to uracil; and
detecting the oligonucleotide, thereby identifying the target in the mixture.

16. The method of claim 15, wherein the converting step is selected from adding bisulfite ions to the mixture and exposing the mixture to cytosine deaminase.

17. The method of claim 15, where the oligonucleotide is DNA.

18. The method of claim 17, wherein the detecting step comprises amplifying DNA molecules that do not contain uracil.

19. The method of claim 17, wherein the detecting step comprises degrading DNA molecules that contain uracil.

20. The method of claim 15, wherein the nucleic acid molecules are mRNA.

21. A method of making a library of targets from a mixture of RNA molecules, the method comprising:
providing the mixture of RNA molecules;
adding to the mixture a plurality of non-naturally-occurring oligonucleotides, each oligonucleotide comprising:
a common 5′ region free of cytosines;
a central region comprising at least one cytosine and complementary to a portion of a target; and
a common 3′ region free of cytosines,
such that at least one of the plurality of oligonucleotides anneals with at least one target, thereby forming a base pair between the at least one cytosine and at least one guanine in the target;
converting unpaired cytosines in the mixture to uracil; and
selecting oligonucleotides that do not contain uracil for making the library.

22. The method of claim 21, wherein the converting step is selected from adding bisulfite ions to the mixture and exposing the mixture to cytosine deaminase.

23. The method of claim 21, wherein the oligonucleotides are DNA.

24. The method of claim 23, wherein the selecting step comprises amplifying DNA molecules that do not contain uracil.

25. The method of claim 23, wherein the selecting step comprises degrading DNA molecules that contain uracil.

26. The method of claim 21, wherein the RNA molecules are mRNA.
